(19) United States
(12) Patent Application Publication
(10) Pub. No.: US 2020/0150025 A1
(43) Pub. Date: May 14, 2020

(54) METHODS FOR EVALUATING CARBON STEELS

(71) Applicant: ExxonMobil Research and Engineering Company, Annandale, NJ (US)

(72) Inventors: Fang Cao, Basking Ridge, NJ (US); Yao Xiong, Cypress, TX (US); David S. Fischer, Houston, TX (US); Jorge L. Pacheco, Houston, TX (US)

(21) Appl. No.: 16/660,880

(22) Filed: Oct. 23, 2019

Related U.S. Application Data
(60) Provisional application No. 62/760,324, filed on Nov. 13, 2018.

(51) Int. Cl. G01N 17/00; G01N 23/2251; G01N 33/204; C22C 38/00

(52) U.S. Cl. CPC G01N 17/006; G01N 23/2251; G01N 2223/418; C22C 38/00; G01N 33/204

(57) ABSTRACT

Carbon steels are desirable for many applications due to their hardness and low cost. Susceptibility toward corrosion can complicate the use of carbon steels, and there is presently no ready way to predict susceptibility toward corrosion for a given carbon steel under a particular set of corrosive conditions. Methods for predicting susceptibility toward corrosion and determining the suitability of a carbon steel may comprise: obtaining a group of carbon steel samples, each carbon steel sample having an unknown rate of corrosion and a microstructure; measuring a quantity of carbides within the microstructure of each carbon steel sample having an unknown rate of corrosion; and determining a relative order of susceptibility toward corrosion within the group of carbon steel samples based upon the quantity of carbides within the microstructure of each carbon steel sample.

### CROSS-REFERENCE TO RELATED APPLICATIONS

This application claims priority to U.S. Provisional Application Serial No. 62/760,324 filed Nov. 13, 2018, which is herein incorporated by reference in its entirety.

### FIELD

The present disclosure relates to corrosion of carbon steels and approaches for evaluating susceptibility of carbon steels toward corrosion.

### BACKGROUND

Carbon steel comprises predominantly iron in combination with varying amounts of to carbon and optional alloying elements. The amounts of carbon and other alloying elements may be chosen to convey particular properties to a given carbon steel. Up to about 2.1 wt. % carbon may be present in carbon steel. As defined by the American Iron and Steel Institute, a given steel is considered to be carbon steel when the specified minimum content for copper does not exceed 0.40 wt. % and the specified maximum contents for manganese, silicon, and copper do not exceed 1.65 wt. %, 0.60 wt. %, and 0.60 wt. %, respectively. No minimum content is specified or required for other alloying elements.

Carbon steel formulation and manufacturing often require a delicate act of simultaneously balancing various chemical and mechanical properties to promote suitability for a given application. The amounts of carbon and other alloying elements contribute considerably to the chemical and mechanical properties of carbon steel. In addition, the particular conditions under which processing of a carbon steel takes place may further determine the ultimate chemical and mechanical properties that are obtained.

Carbon steels may be further categorized based upon their chemical and/or physical properties, with the categorization frequently being utilized to guide selection of a carbon steel for use in a particular application. For example, certain grades of carbon steel may be categorized based on their mechanical strength or on their amount of added corrosion-suppressing alloying elements (e.g., chromium is frequently added for its corrosion-suppressing effects). Carbon steels suitable for high yield seamless or welded pipe applications, such as those used in the oil and gas industries, are frequently characterized as American Petroleum Institute (API) 5L “X” grades (e.g., X42, X46, X52, X56, X60, X65, X70, and X80), where the number following the “X” represents the minimum yield strength (in thousands of psi) for the particular carbon steel. Other mechanical properties, such as tensile strength and/or elongation, may also be used to categorize carbon steels and guide one's selection of a carbon steel for a particular application. Another category of API steels includes those of the L80 class, which are controlled yield strength carbon steels having enhanced corrosion resistance for use in sour (i.e., H₂S-containing) wells. When categorized based upon a particular classifying property, the various carbon steels within a given category may exhibit a wide range of elemental compositions and microstructural variation. Other non-categorized properties within a given category may also vary considerably. As such, when choosing a carbon steel based upon a specified categorizing property, further consideration may need to be given to the particular environment to which the carbon steel will be exposed to account for variations in chemical composition and microstructure.

Carbon steels have been widely used in the oil and gas industry due to their low cost. Selection of a carbon steel for a given application is oftentimes based upon mechanical and physical properties, as well as fabrication suitability (e.g., weldability, machinability, and formability). Corrosion resistance is not a property that is usually associated with carbon steels, given their high iron content. Both uniform thinning (i.e., general corrosion) and localized pitting are encompassed herein by the broad term “corrosion.” Although certain alloying elements, such as chromium, may be introduced into a carbon steel to suppress corrosion, exposure of a carbon steel to a corrosive environment still requires a knowledge of the rate at which the carbon steel undergoes corrosion under the particular corrosive conditions that are present therein. At present, there is no ready way to determine how a given carbon steel will behave upon exposure to particular corrosive conditions. Time-consuming laboratory corrosion tests are currently performed in order to identify a carbon steel having potential suitability for a specified application.

### SUMMARY

In various embodiments, the present disclosure provides methods for predicting susceptibility of carbon steels toward corrosion based upon a quantity of carbides therein. The methods comprise: obtaining a group of carbon steel samples, each carbon steel sample having an unknown rate of corrosion and a microstructure; measuring a quantity of carbides within the microstructure of each carbon steel sample; and determining a relative order of susceptibility toward corrosion within the group of carbon steel samples based upon the quantity of carbides within the microstructure of each carbon steel sample. The carbon steel samples may be exposed to corrosive conditions in which a protective corrosion scale is not formed.

In other various embodiments, the present disclosure provides methods for predicting susceptibility of carbon steels toward corrosion based upon total carbon content. The methods comprise: obtaining a group of carbon steel samples, each carbon steel sample having an unknown rate of corrosion and a thermodynamically stable microstructure; and determining a relative order of susceptibility toward corrosion within the group of carbon steel samples based upon a total carbon content of each carbon steel sample. The carbon steel samples may be exposed to corrosive conditions in which a protective corrosion scale is not formed.

In still other various embodiments, the present disclosure provides methods for identifying a carbon steel suitable for use under a particular set of corrosive conditions. The methods comprise: identifying or providing a set of corrosive conditions; obtaining or measuring a rate of corrosion under the set of corrosive conditions for one or more standard carbon steel samples, each standard carbon steel sample having a known quantity of carbides; obtaining or preparing a carbon steel having an unknown rate of corrosion under the set of corrosive conditions; and determining a relative susceptibility toward corrosion or a rate of corrosion for the carbon steel under the set of corrosive conditions by comparing a quantity of carbides therein to known quantity of carbides in the one or more standard carbon steel samples and the rates of corrosion for the one or more standard carbon steel samples. The carbon steel samples may be exposed to corrosive conditions in which a protective corrosion scale is not formed.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one of ordinary skill in the art and having the benefit of this disclosure.

FIGS. 1A-1E show illustrative low-resolution SEM or STEM images of the carbon steels from Entries 1-5 in the Examples, respectively. FIGS. 1F-1J show corresponding high-resolution SEM or STEM images of the carbon steels.

FIGS. 2A-2C show illustrative SEM images of the various X52 carbon steels from Entries 6-8 in the Examples, respectively. FIGS. 2D-2F show corresponding optical microscopy images of the carbon steels.

FIG. 3 shows a plot of total carbon content versus volume percentage of carbides for the carbon steel samples tested in the Examples herein.

FIG. 4 shows a plot of B scale Rockwell Hardness versus the measured volume percentage of carbides for the carbon steel samples tested in the Examples herein.

FIG. 5 shows a plot of volume percentage of carbides versus the weight loss corrosion rate for the carbon steel samples of Entries 1-5 in the Examples.

FIG. 6 shows a plot of volume percentage of carbides versus the weight loss corrosion rate for the carbon steel samples of Entries 1-5 in the Examples in the presence of a corrosion inhibitor.

FIG. 7 shows a plot of volume percentage of carbides versus the weight loss corrosion rate for the carbon steels of Entries 6-8 in the Examples, both in the presence of and in the absence of a corrosion inhibitor.

FIG. 8 shows a plot of total carbon content versus volume percentage of carbides for the X52 carbon steel samples tested in Example 2.

FIG. 9 shows the weight loss corrosion rate data of FIG. 7 replotted against the total carbon content of the X52 carbon steel samples.

FIG. 10 shows the weight loss corrosion rate data of Examples 1 and 2 replotted against B scale Rockwell hardness values.

FIG. 11 shows the weight loss corrosion rate data versus the volume percentage of carbides for carbon steel samples at different pH values.

### DETAILED DESCRIPTION

The present disclosure generally relates to methods for selecting a carbon steel based upon a quantity of carbides within the microstructure of the carbon steel.

As discussed above, carbon steels may be categorized based upon their mechanical properties, which may be used to guide selection of a particular carbon steel for applicability in a given application. A wide range of carbon steel formulations and microstructures may be present within a given carbon steel category. As such, even though similarly categorized carbon steels may share one or more properties in common, there can be considerable variation in other properties due to the breadth of carbon steel formulations and microstructures that may be present. Different processing conditions may similarly alter the properties of carbon steels, even for carbon steels having similar formulations.

One factor that may vary considerably within similarly categorized carbon steels is the rate of corrosion under a particular set of corrosive conditions. At present, there is no generalized way to predict the absolute rate of corrosion for a given carbon steel or the relative rates of corrosion within a particular group of carbon steels. Time-consuming laboratory testing is usually performed to determine the rate of corrosion for one or more carbon steels to evaluate suitability for a given application. Even then, laboratory-measured rates of corrosion may not correlate to corrosion rates observed in the field.

The carbon content of carbon steels may be present in several morphological forms. One form may be that of “soluble” carbon, which is present in a solid solution with iron. The solubility of carbon is limited in ferrite phases (negligible solubility at room temperature) but is considerably higher in austenite and martensite phases. Certain alloying elements may also promote or stabilize the formation of an austenite phase to improve the solubility of carbon. Another form of carbon within carbon steels is that of metal carbides, which may be formed with iron or one or more of the alloying elements. Mixtures of soluble carbon and metal carbides are frequently present in a carbon steel sample. The reported total carbon content of a carbon steel sample is generally reported as the sum of the carbon contributions from carbon in solid solution and metal carbides. The ratio between soluble carbon and metal carbides may be impacted, for example, by the processing conditions of the carbon steel (e.g, heating and cooling rates during processing), the amount of carbon that is present, and/or the presence or absence of various alloying elements. Certain iron allotropes, such as austenite, may also promote solubilization of carbon rather than formation of metal carbides in various instances.

The present disclosure demonstrates that the quantity of carbides (carbide content) in carbon steels, particularly metal carbides residing at the grain boundaries and within the grains in the microstructure of the carbon steels, may he correlated to a rate of corrosion. The carbide contents of each member of a group of carbon steel samples having unknown rates of corrosion may be used to determine a relative order of susceptibility of the carbon steel samples toward corrosion. Moreover, by comparing one or more carbon steel samples having an unknown rate of corrosion against standard carbon steel samples having a known or measured carbide content and a known or measured rate of corrosion under a particular set of corrosive conditions, the corrosion rate(s) of the one or more carbon steel samples having an unknown rate of corrosion may be predicted, at least semi-quantitatively. Thus, the present disclosure provides ready techniques for identifying a particular carbon steel from among a group of carbon steels, including groups having carbon steels with at least one shared property in common, based upon an anticipated rate of corrosion under a particular set of corrosive conditions. The concepts of the present disclosure may he particularly applicable in environments where there is no protective corrosion scale formed upon a surface of the carbon steel.

Advantageously, the carbon steel selection techniques disclosed herein may be employed in lieu of laboratory corrosion testing, thereby decreasing the time needed to choose a carbon steel for use in a particular application. Alternately, the carbon steel selection techniques disclosed herein may be used to improve the efficiency of conducting laboratory corrosion testing and possibly decreasing the time needed to do so. Namely, the carbon steel selection techniques disclosed herein may be used to guide laboratory testing of a smaller suite of carbon steels than would otherwise be feasible. Under either scenario, reduced costs for selecting a suitable carbon steel may be realized.

Before describing the methods of the present disclosure in further detail, a listing of terms follows to aid in better understanding the present disclosure.

All numerical values within the detailed description and the claims herein are modified by “about” or “approximately” with respect to the indicated value, and take into account experimental error and variations that would be expected by a person having ordinary skill in the art. Unless otherwise indicated, room temperature is about 25° C.

As used in the present disclosure and claims, the singular forms “a,” “an,” and “the” include plural forms unless the context clearly dictates otherwise.

The term “and/or” as used in a phrase such as “A and/or B” herein is intended to include “A and B,” “A or B,” “A”, and “B.”

For the purposes of the present disclosure, the new numbering scheme for groups of the Periodic Table is used. In said numbering scheme, the groups (columns) are numbered sequentially from left to right from 1 through 18, excluding the f-block elements (lanthanides and actinides).

The term “corrosion resistance” refers to a material's propensity to deteriorate upon exposure to a reactive or corrosive environment.

The term “rate of corrosion” refers to the amount of material loss (mass loss) from a body per unit time upon exposure to a reactive or corrosive environment, also referred to as corrosive conditions. The term “rate of corrosion” may be used synonymously with the term “absolute corrosion rate” or “absolute rate of corrosion.”

The term “relative susceptibility toward corrosion” refers to the rate of corrosion of one body in comparison to one or more other bodies, without reference to the absolute rates of corrosion, such as the rates of corrosion of one or more carbon steel samples in comparison to one another.

The term “relative order of susceptibility toward corrosion” refers to a ranking of the rates of corrosion with a group, such as a group of carbon steel samples.

The term “toughness” refers to a material's resistance to crack initiation and propagation.

The term “yield strength” refers to a material's ability to bear a load without deformation.

The term “tensile strength” refers to the strength corresponding to the maximum load carrying capability of a material in units of stress when the failure mechanism of the material is not linear elastic fracture.

The term “austenite,” also referred to as “gamma iron,” refers to an iron allotrope or an iron/alloying element solid solution having a face-centered cubic (FCC) atomic crystalline structure. Austenite may display a higher propensity for dissolving carbides than does ferrite.

The term “ferrite,” also referred to as “alpha iron,” refers to an iron allotrope having a body-centered cubic (BCC) atomic crystalline structure. Ferrite has a poor propensity for dissolving carbides, particularly when compared to austenite.

The term “carbide” refers to a compound of carbon with iron, another metal element, or a non-metal element.

The term “cementite” refers to an iron carbide compound, particularly the iron carbide compound having the formula Fe₃C.

The term “pearlite” refers to a lamellar structure comprising alternating layers of ferrite and cementite.

The term “martensite” refers to a steel metallurgical phase obtained from rapid quenching of austenite.

According to some embodiments, the present disclosure provides methods for determining the relative order of susceptibility toward corrosion within a group of carbon steels. More specifically, such methods may comprise obtaining a group of carbon steel samples, in which each carbon steel sample has an unknown rate of corrosion and a microstructure; measuring a quantity of carbides within the microstructure of each carbon steel sample; and determining a relative order of susceptibility toward corrosion within the group of carbon steel samples based upon the quantity of carbides within the microstructure of each carbon steel sample.

In various embodiments of the present disclosure, the quantity of carbides in the carbon steels or carbon steel samples may be determined by analyzing (e.g., via image processing) a scanning electron micrograph (SEM), scanning transmission electron micrograph (STEM) or optical micrograph (OM) using appropriate image processing software, such as ImageJ (public domain software). Other image processing software may be used similarly to determine the quantity of carbides. To determine the quantity of carbides, an average may be taken following measurement of a predetermined number of samples or a predetermined number of measurements upon a particular sample (e.g., 5-6).

Methods of the present disclosure may allow one to determine which carbon steel samples within a group are more prone to corrosion than are other group members. Thus, the present disclosure provides for determining a relative ordering of corrosion rates within a group of carbon steel samples. In particular, according to the present disclosure, carbon steel samples containing a larger quantity of carbides are expected to undergo corrosion at a faster rate relative to those having a smaller quantity of carbides. The absolute corrosion rate need not necessarily be determined, for example, if a qualitative evaluation of relative susceptibility toward corrosion is sufficient to characterize a group, particularly to choose a carbon steel sample from within the group for a particular application. For example, one may choose a carbon steel sample having one or more predetermined mechanical properties and that is likely to undergo corrosion at the lowest rate during deployment in a corrosive environment. If desired, absolute corrosion rates may he determined through an extension of the foregoing methods, as explained further herein below.

The types of corrosion that may be predicted by the methods described herein is not believed to be particularly limited in terms of the corrosive environment. Corrosive environments may comprise acids, salt water, brine, corrosive gases (e.g., carbon dioxide or hydrogen sulfide), or any combination thereof. In particular embodiments, the relative order of susceptibility toward corrosion may be determined for “sweet” corrosive environments comprising carbon dioxide or “sour” corrosive environments comprising hydrogen sulfide. Absolute corrosion rates may be determined in the foregoing types of corrosive environments as well.

The methods described herein may he particularly applicable in corrosive environments where there is no protective corrosion scale formed upon a surface of the carbon steel samples, Protective scales that may form under certain conditions when carbon steels are exposed to a corrosive environment include, for example, iron carbonate or iron sulfide, depending upon whether sweet or sour corrosive conditions are present. Iron carbonate, for example, may form at a pH of about 6 or greater at a temperature of about 65° C. or greater.

The carbon steel samples within the group of carbon steels are not necessarily all of the same functional category (e.g., characterized by a particular mechanical property) or even of similar chemical compositions and/or microstructures. Iron and carbon are required elements in carbon steels by definition. Additional elements that may be present in the carbon steel samples in order to tailor their mechanical and/or chemical properties, including rates of corrosion include, for example, Mn, Si, Ni, Cr, Mo, Cu, Al, V, Nb, B, Ti, N, Co, Ce, Sn, Zn, Pb, Zr and any combination thereof It is also to be appreciated that various low-level manufacturing impurities may also be present, sometimes depending upon which additional elements are present and the chosen processing conditions for a given steel sample. The amounts of the additional elements and associated processing conditions for the steel samples are not considered to be particularly invited when practicing the methods of the present disclosure. A detailed discussion of the anticipated. function of each of the additional elements that may be included in a carbon steel sample is beyond the scope of the present disclosure. However, one having ordinary skill in the art will be able to appreciate the anticipated function of including particular additional elements in specified amounts and/or ratios. Moreover, one having ordinary skill in the art will be able to appreciate the effects that various processing conditions may convey to a carbon steel sample. By determining the amount of carbides in carbon steel samples according to the disclosure herein, other factors such as chemical, microstructural, and mechanical property variations may be largely dismissed with respect to their corrosion-promoting effects.

The carbon steels within the group of carbon steel samples may all have the same type of microstructure (e.g., a ferrite/pearlite microstructure), or two or more different types of microstructures may be present in the group of carbon steel samples. Advantageously, the methods of the present disclosure are not dependent upon all of the carbon steel samples in the group having the same microstructure. Thus, the particular microstructural environment in which the quantity of carbides is located does not appreciably impact the relative order of susceptibility toward corrosion and/or the predicted corrosion rate under a particular set of corrosive conditions. Certain correlations of the total carbon content (Cₜₒₜₐₗ) to the rate of corrosion may be made in some cases where the microstructures are of the same type, however, as discussed in more detail below.

Methods of the present disclosure may further comprise determining an absolute rate of corrosion for at least one of the carbon steel samples within the group of carbon steel samples, as described in further detail hereinafter. For example, in some embodiments, it may be of interest to determine the absolute rate of corrosion for the carbon steel sample predicted to exhibit the lowest relative susceptibility toward corrosion. Determining the absolute rate of corrosion under a particular set of corrosive conditions may aid in evaluating whether the particular carbon steel sample is indeed suitable for use in the corrosive conditions.

The amount of carbides within one or more of the carbon steel samples in the group may be compared to one or more standard carbon steel samples having a known or measured quantity of carbides therein. The one or more standard carbon steel samples may have a known rate of corrosion under a particular set of corrosive conditions, or the rate of corrosion may be measured de novo under the particular set of corrosive conditions. In either case, the rate of corrosion and the quantity of carbides in the one or more standard carbon steel samples may be used to determine a rate of corrosion for one or more of the carbon steel samples having an unknown rate of corrosion. Thus, the methods of the present disclosure may allow ready determination of predicted rates of corrosion for a large number of carbon steel samples without having to perform lengthy corrosion testing thereon. At the very least, such methods may aid in identifying one or more candidate carbon steel samples for conducting more detailed laboratory corrosion testing thereon. Similarly, the methods of the present disclosure may guide formulation or processing of a carbon steel, such that a higher or lower quantity of carbides is formed therein to promote a desired rate of corrosion in a particular corrosive environment.

In more specific embodiments, such methods may comprise: identifying or providing a set of corrosive conditions; measuring a rate of corrosion under the set of corrosive conditions for one or more standard carbon steel samples, each standard carbon steel sample having a known or measured quantity of carbides; and determining a rate of corrosion under the set of corrosive conditions for one or more of the carbon steel samples having the unknown rate of corrosion by comparing the quantity of carbides therein to the quantity of carbides in the one or more standard carbon steel samples and the rates of corrosion under the set of corrosive conditions for the one or more standard carbon steel samples. Alternately, such methods may comprise: identifying or providing a set of corrosive conditions; providing one or more standard carbon steel samples, each standard carbon steel sample having a known or measured rate of corrosion under the set of corrosive conditions and a known or measured quantity of carbides; and determining a rate of corrosion under the set of corrosive conditions for one or more of the carbon steel samples having the unknown rate of corrosion by comparing the quantity of carbides therein to the quantity of carbides in the one or more standard carbon steel samples and the rates of corrosion under the set of corrosive conditions for the one or more standard carbon steel samples. As mentioned above, corrosive conditions are not considered to be particularly limited in the methods of the present disclosure, and may be sweet corrosive conditions in particular embodiments. Other corrosive conditions may be those in which no protective corrosion scale is formed upon a surface of the carbon steel samples.

Comparison between the one or more carbon steel samples having an unknown rate of corrosion and the one or more standard carbon steel samples may take place by any suitable means. In more particular embodiments, the known or measured rate of corrosion and the known or measured quantity of carbides for each of the one or more standard carbon steel samples may be populated in a lookup table, fit to a calibration function (e.g., the rate of corrosion as a function of the quantity of carbides present in the carbon steel sample), plotted in a calibration curve (e.g., a plot having the quantity of carbides on one axis and the rate of corrosion on the other axis), or any combination thereof. The rate of corrosion under the set of corrosive conditions for one or more of the carbon steel samples, each having an unknown rate of corrosion, may then be determined based upon the quantity of carbides within the microstructure of the one or more carbon steel samples, and consulting the lookup table, the calibration function, the calibration curve, or any combination thereof,

Comparison of the quantity of carbides in the one or more carbon steel samples to the lookup table, the calibration function, and/or the calibration curve may be performed by various methods that will be familiar to one having ordinary skill in the art. Lookup tables suitable for use in the disclosure herein may be in hard copy or electronic form (e.g., a database or spreadsheet). Suitable lookup tables may feature a plurality of entries including a first column containing various amounts of carbides (e.g., insoluble carbides) and a second column having corresponding rates of corrosion for each of the various amounts of carbides. The lookup table may be populated before evaluating a carbon steel sample having an unknown rate of corrosion. The lookup table may then be consulted, either manually or automatically using a suitable processor and user interface, to determine which entry in the lookup table contains a quantity of carbides that is closest to that measured for the carbon steel sample having an unknown rate of corrosion. Once the correct entry in the lookup table has been found, the corresponding rate of corrosion for the carbon steel sample having an unknown rate of corrosion may then be determined. In some cases, interpolation between entries in the lookup table may be conducted, such as, for example, when the quantity of carbides in the carbon steel sample having an unknown rate of corrosion differs from the values in the lookup table by a predetermined amount. Interpolation may assume a linear variance in the rate of corrosion between the measured quantities of carbides for the one or more standard carbon steel samples.

Like a lookup table, a calibration function or a calibration curve (plot) may be determined before evaluating a carbon steel sample having an unknown rate of corrosion. The calibration function or calibration curve may be determined by curve fitting or plotting measured rates of corrosion against corresponding quantities of carbides for each of the standard carbon steel samples. The curve fit may be a linear or non-linear fitting function. Once determined, the calibration curve or calibration function may be consulted, either manually or automatically using a suitable processor and user interface, to determine an anticipated rate of corrosion for one or more carbon steel samples having an unknown rate of corrosion.

According to more specific embodiments, the carbides used for determining the relative susceptibility toward corrosion and/or the rate of corrosion for the carbon steel samples having the unknown rate of corrosion represents one or more metal carbides that are located at grain boundaries or within grains that are present within the microstructure of the carbon steel samples. The metal carbides may be an iron carbide (e.g., cementite) or a carbide of one or more of the additional elements comprising the carbon steel samples.

In still more specific embodiments, the quantity of carbides in the carbon steel samples (e.g., carbides located at or within the grain boundaries) may be determined via imaging techniques, such as by scanning electron microscopy or scanning transmission electron microscopy. Image processing software, such as ImageJ, for example, may be used for analyzing the carbon steel images to determine the quantity of carbides that are present therein.

Measurement of the relative order of susceptibility toward corrosion and/or the rate of corrosion under a particular set of corrosive conditions may be used to determine the suitability of a carbon steel for particular deployment conditions and deployment times in a given corrosive environment. More specifically, the methods may comprise, identifying or providing a corrosive work environment, and selecting a carbon steel from among the group of carbon steel samples to comprise a workpiece for deployment in the corrosive work environment based upon an anticipated deployment time of the workpiece in the corrosive work environment, and a predicted susceptibility toward corrosion or a predicted rate of corrosion of the carbon steel in the corrosive work environment. The predicted susceptibility toward corrosion or the predicted rate of corrosion may be determined based upon the quantity of carbides within the microstructure of the carbon steel, as discussed herein. Suitable workpieces are not considered to be particularly limited and may include, for example, tools or objects inserted in a corrosive work environment (e.g., downhole tools), a pipeline or similar conduit through which the corrosive work environment flows, or any combination thereof

As mentioned above, a correlation between the total carbon content (Cₜₒₜₐₗ) and the quantity of carbides in the carbon steel samples may be made when the microstructures are all thermodynamically stable, such that there is no intercon version between carbon in solid solution and metal carbides. For example, when the microstructure of the carbon steels is a thermodynamically stable ferrite/pearlite microstructure, there is no interconversion between the various forms of carbon. Thus, in such instances, the total carbon content of the carbon steel samples correlates directly with the quantity of carbides therein, and the predicted susceptibility toward corrosion and/or the predicted rate of corrosion may likewise be correlated with the total carbon content. Correlation of the total carbon content to the susceptibility toward corrosion and/or the rate of corrosion may be accomplished using similar comparison techniques to those discussed above,

Accordingly, some embodiments of the present disclosure may comprise: obtaining a group of carbon steel samples, each carbon steel sample having an unknown rate of corrosion and a thermodynamically stable microstructure; and determining a relative order of susceptibility toward corrosion within the group of carbon steel samples based upon a total carbon content of each carbon steel sample. The thermodynamically stable microstructures may be compositionally the same or compositionally different, according to various embodiments.

As in the related embodiments discussed above, the total carbon content may be further used to determine a rate of corrosion for one or more carbon steel samples under particular corrosive conditions. Accordingly, in some embodiments, methods of the present disclosure may further comprise: identifying or providing a set of corrosive conditions; measuring a rate of corrosion under the set of corrosive conditions for one or more standard carbon steel samples, in which each standard carbon steel sample has a known or measured total carbon content and a thermodynamically stable microstructure; and determining a rate of corrosion under the set of corrosive conditions for one or more of the carbon steel samples having the unknown rate of corrosion by comparing the total carbon content within each of the carbon steel samples to the total carbon content in the one or more standard carbon steel samples and the rates of corrosion under the set of corrosive conditions for the one or more standard carbon steel samples. Alternately, methods of the present disclosure may further comprise: identifying or providing a set of corrosive conditions; providing one or more standard carbon steel samples, in which each standard carbon steel sample has a known or measured total carbon content, a known or measured rate of corrosion under the set of corrosive conditions, and a thermodynamically stable microstructure; and determining a rate of corrosion under the set of corrosive conditions for one or more of the carbon steel samples having the unknown rate of corrosion by comparing the quantity of total carbon within each of the carbon steel samples to the total carbon content in the one or more standard carbon steel samples and the rates of corrosion under the set of corrosive conditions for the one or more standard carbon steel samples.

Similar to the embodiments discussed above, the total carbon content and the rates of corrosion for the one or more standard carbon steel samples may be populated in a lookup table, tit to a calibration function, plotted in a calibration curve, or any combination thereof. Based upon the total carbon content in the one or more carbon steels within the group of carbon steels and by consulting the lookup table, the calibration function, and/or the calibration curve, the rate of corrosion under the set of corrosive conditions may be determined, at least semi-quantitatively, for the one or more carbon steel samples.

Likewise, methods for correlating total carbon content in the carbon steel samples to determine susceptibility toward corrosion and/or the rate of corrosion to ascertain suitability for use of a carbon steel within a corrosive work environment may comprise: identifying a corrosive work environment; and selecting a carbon steel to comprise a workpiece for deployment in the corrosive work environment based upon an anticipated deployment time of the workpiece in the corrosive work environment, and a predicted susceptibility toward corrosion or a predicted rate of corrosion in the corrosive work environment, in which the predicted susceptibility toward corrosion or the predicted rate of corrosion is determined based upon the total carbon content of the carbon steel.

According to more specific embodiments, the carbon steel samples having the unknown rate of corrosion may all have the same type of thermodynamically stable microstructure. In some or other more specific embodiments, the standard carbon steel samples may all have the same type of thermodynamically stable microstructure, and the thermodynamically stable microstructure of the carbon steel samples may be the same as that within the carbon steel samples having the unknown rate of corrosion.

Suitability of a carbon steel for use in a corrosive work environment based upon the total carbon content of the carbon steel may also be determined using the methods disclosed herein In various embodiments, such methods may comprise: identifying or providing a corrosive work environment; and selecting a carbon steel from among the group of carbon steel samples to comprise a workpiece for deployment in the corrosive work environment based upon an anticipated deployment time of the workpiece in the corrosive work environment and a predicted susceptibility toward corrosion or a predicted rate of corrosion of the carbon steel in the corrosive work environment, in which the predicted susceptibility toward corrosion or the predicted rate of corrosion is determined based upon the total carbon content of the carbon steel.

In still other various embodiments, methods of the present disclosure may comprise determining a relative susceptibility toward corrosion and/or a rate of corrosion for a sourced (obtained) or prepared (synthesized) carbon steel. More specifically, such methods may comprise: identifying or providing a set of corrosive conditions; obtaining or measuring a rate of corrosion under the set of corrosive conditions for one or more standard carbon steel samples, each standard carbon steel sample having a known quantity of carbides; obtaining or preparing a carbon steel having an unknown rate of corrosion under the set of corrosive conditions; and determining a relative susceptibility toward corrosion or a rate of corrosion for the carbon steel sample under the set of corrosive conditions by comparing a quantity of carbides therein to the known quantity of carbides in the one or more standard carbon steel samples and the rates of corrosion for the one or more standard carbon steel samples. Thus, the present disclosure may be used to guide formulation and/or processing of a carbon steel to provide a quantity of carbides sufficient to afford a desired rate of corrosion in the carbon steel.

The quantity of carbides in the carbon steel having an unknown rate of corrosion may be previously known or unknown. If unknown, methods of the present disclosure may comprise measuring the quantity of carbides in the carbon steel prior to determining a rate of corrosion for the carbon steel. Suitable techniques for measuring the carbides within the carbon steel are provided hereinabove. In particular embodiments, the quantity of carbides in the carbon steel sample may he measured via imaging using scanning electron microscopy, scanning transmission electron microscopy, or optical microscopy, for example.

Embodiments disclosed herein include:

A. Methods for determining susceptibility of carbon steel toward corrosion. The methods comprise: obtaining a group of carbon steel samples, each carbon steel sample having an unknown rate of corrosion and a microstructure; measuring a quantity of carbides within the microstructure of each carbon steel sample; and determining a relative order of susceptibility toward corrosion within the group of carbon steel samples based upon the quantity of carbides within the microstructure of each carbon steel sample.

B. Methods for determining susceptibility of carbon steel having a thermodynamically stable microstructure toward corrosion. The methods comprise: obtaining a group of carbon steel samples, each carbon steel sample having an unknown rate of corrosion and a thermodynamically stable microstructure; and determining a relative order of susceptibility toward corrosion within the group of carbon steel samples based upon a total carbon content of each carbon steel sample.

C. Methods for determining susceptibility of a carbon steel sample toward corrosion. The methods comprise: identifying or providing a set of corrosive conditions; obtaining or measuring a rate of corrosion under the set of corrosive conditions for one or more standard carbon steel samples, each standard carbon steel sample having a known quantity of carbides; obtaining or preparing a carbon steel having an unknown rate of corrosion under the set of corrosive conditions; and determining a relative susceptibility toward corrosion or a rate of corrosion for the carbon steel under the set of corrosive conditions by comparing a quantity of carbides therein to the known quantity of carbides in the one or more standard carbon steel samples and the rates of corrosion for the one or more standard carbon steel samples.

Embodiments A-C may have one or more of the following additional elements in any combination:

Element 1: wherein the relative order of susceptibility toward corrosion is determined with respect to an environment where there is no protective corrosion scale formed upon a surface of the carbon steel samples.

Element 2: wherein the method further comprises: identifying or providing a set of corrosive conditions; measuring a rate of corrosion under the set of corrosive conditions for one or more standard carbon steel samples, each standard carbon steel sample having a known or measured quantity of carbides; and determining a rate of corrosion under the set of corrosive conditions for one or more of the carbon steel samples having the unknown rate of corrosion by comparing the quantity of carbides therein to the quantity of carbides in the one or more standard carbon steel samples and the rates of corrosion under the set of corrosive conditions for the one or more standard carbon steel samples.

Element 3: wherein the method further comprises: identifying or providing a set of corrosive conditions; providing one or more standard carbon steel samples, each standard carbon steel sample having a known or measured rate of corrosion under the set of corrosive conditions and a known or measured quantity of carbides; and determining a rate of corrosion under the set of corrosive conditions for one or more of the carbon steel samples having the unknown rate of corrosion by comparing the quantity of carbides therein to the quantity of carbides in the one or more standard carbon steel samples and the rates of corrosion under the set of corrosive conditions for the one or more standard carbon steel samples.

Element 4: wherein the corrosive conditions are sweet corrosive conditions.

Element 5: wherein the known or measured rate of corrosion and the known or measured quantity of carbides for each of the one or more standard carbon steel samples are populated in a lookup table, fit to a calibration function, plotted in a calibration curve, or any combination thereof, and the rate of corrosion under the set of corrosive conditions for one or more of the carbon steel samples having the unknown rate of corrosion is determined based upon the quantity of carbides within the microstructure and consulting the lookup table, the calibration function, the calibration curve, or any combination thereof

Element 6: wherein the method further comprises: identifying or providing a corrosive work environment; and selecting a carbon steel from among the group of carbon steel samples to comprise a workpiece for deployment in the corrosive work environment based upon an anticipated deployment time of the workpiece in the corrosive work environment, and a predicted susceptibility toward corrosion or a predicted rate of corrosion of the carbon steel in the corrosive work environment, the predicted susceptibility toward corrosion or the predicted rate of corrosion being determined based upon the quantity of carbides within the microstructure of the carbon steel.

Element 7: wherein the carbides are one or more carbides located at grain boundaries or within grains within the microstructure of the carbon steel samples.

Element 8: wherein the quantity of carbides is measured through analyzing an image obtained by optical microscopy, scanning electron microscopy or scanning transmission electron microscopy.

Element 9: wherein the group of carbon steel samples comprises two or more carbon steels having two or more different types of microstructures.

Element 10: wherein the method further comprises: identifying or providing a set of corrosive conditions; measuring a rate of corrosion under the set of corrosive conditions for one or more standard carbon steel samples, each standard carbon steel sample having a known or measured total carbon content and a thermodynamically stable microstructure; and determining a rate of corrosion under the set of corrosive conditions for one or more of the carbon steel samples having the unknown rate of corrosion by comparing the total carbon content within each of the carbon steel samples to the total carbon content in the one or more standard carbon steel samples and the rates of corrosion under the set of corrosive conditions for the one or more standard carbon steel samples.

Element 11: wherein the method further comprises: identifying or providing a set of corrosive conditions; providing one or more standard carbon steel samples, each standard carbon steel sample having a known or measured total carbon content and a known or measured rate of corrosion under the set of corrosive conditions, and having a thermodynamically stable microstructure; and determining a rate of corrosion under the set of corrosive conditions for one or more of the carbon steel samples having the unknown rate of corrosion by comparing the total carbon content within each of the carbon steel samples to the total carbon content in the one or more standard carbon steel samples and the rates of corrosion under the set of corrosive conditions for the one or more standard carbon steel samples.

Element 12: wherein the known or measured rate of corrosion and the total carbon content for each of the one or more standard carbon steel samples are populated in a lookup table, fit to a calibration function, plotted in a calibration curve, or any combination thereof, and the rate of corrosion under the set of corrosive conditions for one or more of the carbon steel samples having the unknown rate of corrosion is determined based upon the total carbon content and consulting the lookup table, the calibration function, the calibration curve, or any combination thereof

Element 13: wherein the standard carbon steel samples all have the same type of thermodynamically stable microstructure and the thermodynamically stable microstructure of the standard carbon steel samples is the same as that within the carbon steel samples having the unknown rate of corrosion.

Element 14: wherein the method further comprises: identifying or providing a corrosive work environment; and selecting a carbon steel from among the group of carbon steel samples to comprise a workpiece for deployment in the corrosive work environment based upon an anticipated deployment time of the workpiece in the corrosive work environment, and a predicted susceptibility toward corrosion or a predicted rate of corrosion of the carbon steel in the corrosive work environment, the predicted susceptibility toward corrosion or the predicted rate of corrosion being determined based upon the total carbon content of the carbon steel.

Element 15: wherein the total carbon content correlates directly with a quantity of carbides within the thermodynamically stable microstructure.

Element 16: wherein the carbon steel samples having the unknown rate of corrosion all have the same type of thermodynamically stable microstructure.

Element 17: wherein the method further comprises: measuring the quantity of carbides in the carbon steel.

By way of non-limiting example, exemplary combinations applicable to the method of A include: elements 1 and 2; 1 and 3; 1 and 4; 1 and 6; 1 and 7; 1 and 8; 1 and 9; 2 or 3 and 4; 2 or 3 and 5; 2 or 3 and 6; 2 or 3 and 7; 2 or 3 and 8; 2 or 3 and 9; 4 and 5; 4 and 6; 4 and 7; 4 and 8; 4 and 9; 5 and 6; 5 and 7; 5 and 8; 5 and 9; 6 and 7; 6 and 8; 6 and 9; 7 and 8; 7 and 9; and 8 and 9.

By way of further non-limiting example, exemplary combinations applicable to the method of B include: elements 1 and 10 or 11; 1 and 12; 1 and 13; 1 and 14; 1 and 15; 1 and 16; 10 or 11 and 12; 10 or 11 and 13; 10 or 11 and 14; 10 or 11 and 15; 10 or 11 and 16; 12 and 13; 12 and 14; 12 and 15; 12 and 16; 13 and 14; 13 and 15; 13 and 16; 14 and 15; 14 and 16; and 15 and 16.

By way of still further non-limiting example, exemplary combinations applicable to the method of C include: elements 1 and 7; 1 and 8; 1 and 17; 7 and 8; 7 and 17; and 8 and 17.

To facilitate a better understanding of the embodiments described herein, the following examples of various representative embodiments are given. In no way should the following examples be read to limit, or to define, the scope of the present disclosure.

### EXAMPLES

Coupons of various carbon steel samples formulated as specified in Table 1 were corrosion tested in the manner described further below. The volume percentage of carbides, also specified in Table 1, in the microstructure of each carbon steel sample was measured by imaging techniques using ImageJ image processing software. Specifically, the volume percentage of carbides in each carbon steel sample was determined by analysis of an optical microscopy image, a scanning electron microscopy image, or a scanning transmission electron microscopy image of the carbon steel sample, in which carbides were visually contrasted from the steel microstructure.

TABLE 1

| | | Vol. % | Wt. % | | | | | | | |
|---|---|---|---|---|---|---|---|---|---|---|
| Entry | Steel | Carbide | Cₜₒₜₐₗ | Mn | Si | Ni | Cr | Mo | Cu | Al |
| 1 | X52 | 35.2 | 0.21 | 0.96 | 0.24 | 0.08 | 0.09 | 0.02 | 0.2 | 0.04 |
| 2 | X60 | 36.1 | 0.06 | 1.61 | 0.09 | 0.22 | 0.02 | <0.01 | 0.16 | <0.01 |
| 3 | X65 | 20.7 | 0.1 | 1.3 | 0.22 | 0.01 | 0.03 | 0.01 | 0.03 | 0.03 |
| 4 | L80 | 14.2 | 0.27 | 1.38 | 0.23 | 0.01 | 0.19 | 0.18 | 0.02 | 0.03 |
| 5 | L801Cr | 34.2 | 0.4 | 0.8 | 0.24 | 0.19 | 1.01 | 0.2 | 0.18 | 0.04 |
| 6 | X52_1 | 2.9 | 0.034 | 1.08 | 0.18 | <0.01 | 0.02 | <0.01 | 0.01 | 0.03 |
| 7 | X52_2 | 34.3 | 0.2 | 0.93 | 0.24 | 0.08 | 0.09 | 0.02 | 0.2 | 0.03 |
| 8 | X52_3 | 46.9 | 0.28 | 1.14 | 0.03 | 0.03 | 0.04 | <0.01 | 0.01 | 0.01 |

| | | Wt. % | | | | |
|---|---|---|---|---|---|---|
| | | | | | | Fe and |
| Entry | Steel | V | Nb | B | Ti | impurities |
| 1 | X52 | 0.022 | <0.008 | <0.005 | <0.008 | balance |
| 2 | X60 | <0.008 | 0.012 | <0.005 | 0.01 | balance |
| 3 | X65 | 0.057 | 0.011 | <0.005 | <0.008 | balance |
| 4 | L80 | <0.008 | <0.008 | <0.005 | <0.008 | balance |
| 5 | L801Cr | <0.008 | <0.008 | <0.005 | <0.008 | balance |
| 6 | X52_1 | <0.008 | 0.016 | <0.005 | 0.009 | balance |
| 7 | X52_2 | 0.021 | <0.008 | <0.005 | <0.008 | balance |
| 8 | X52_3 | 0.95 | <0.008 | <0.005 | <0.008 | balance |

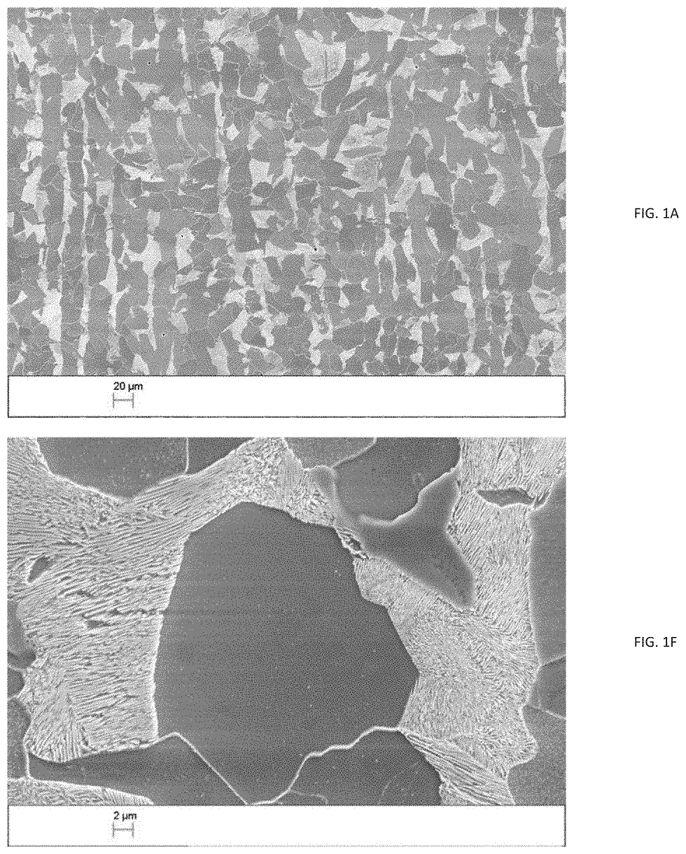

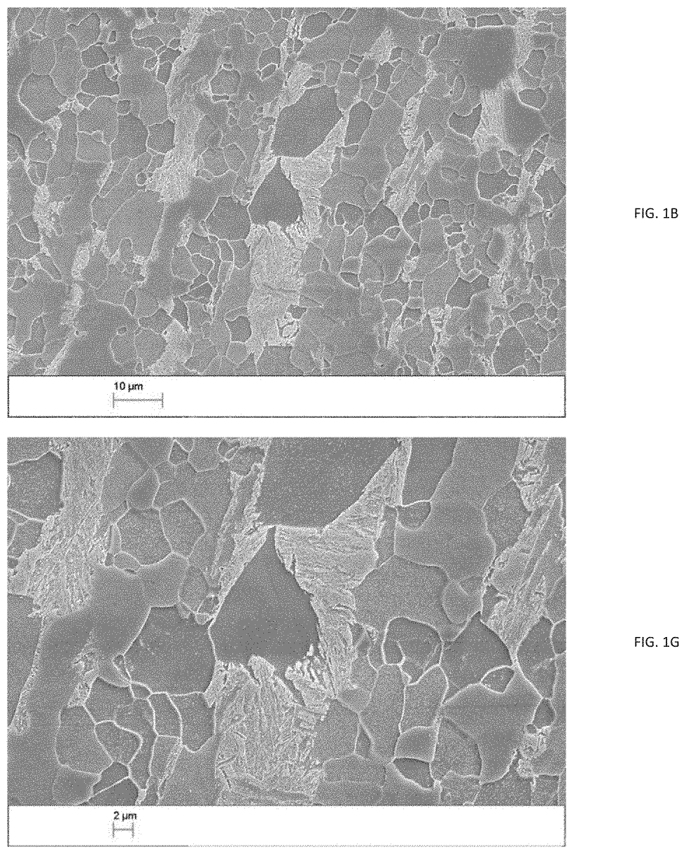

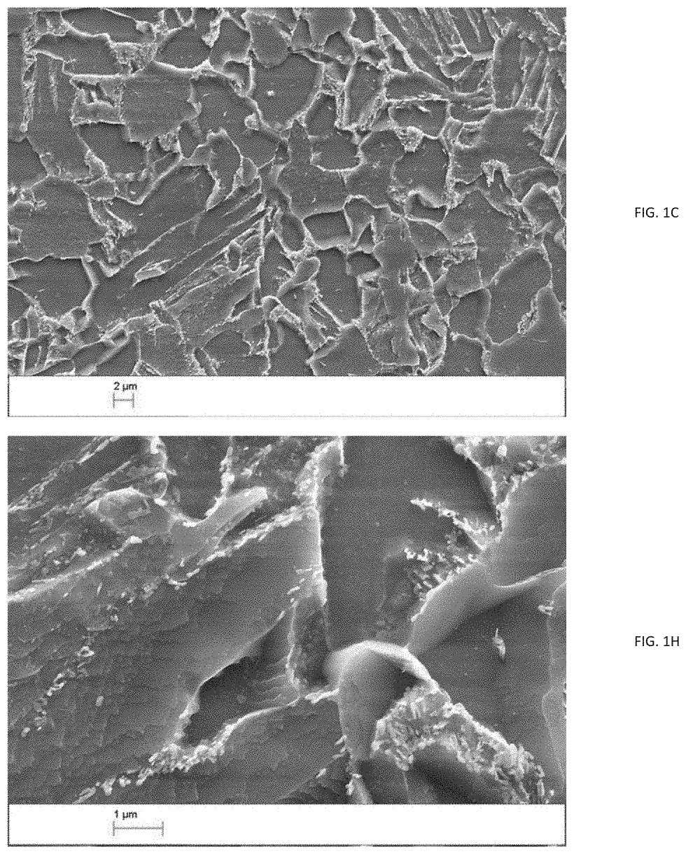

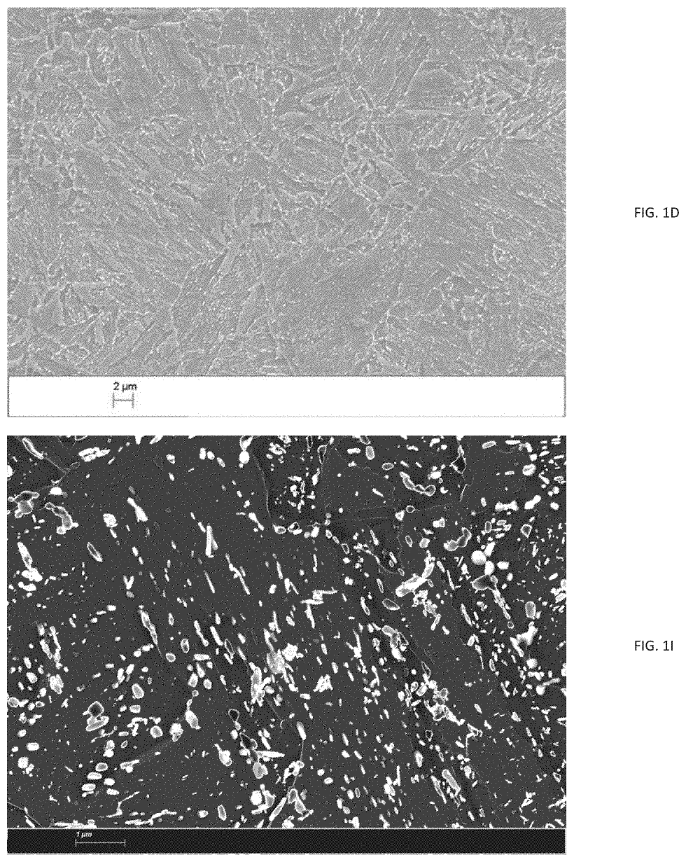

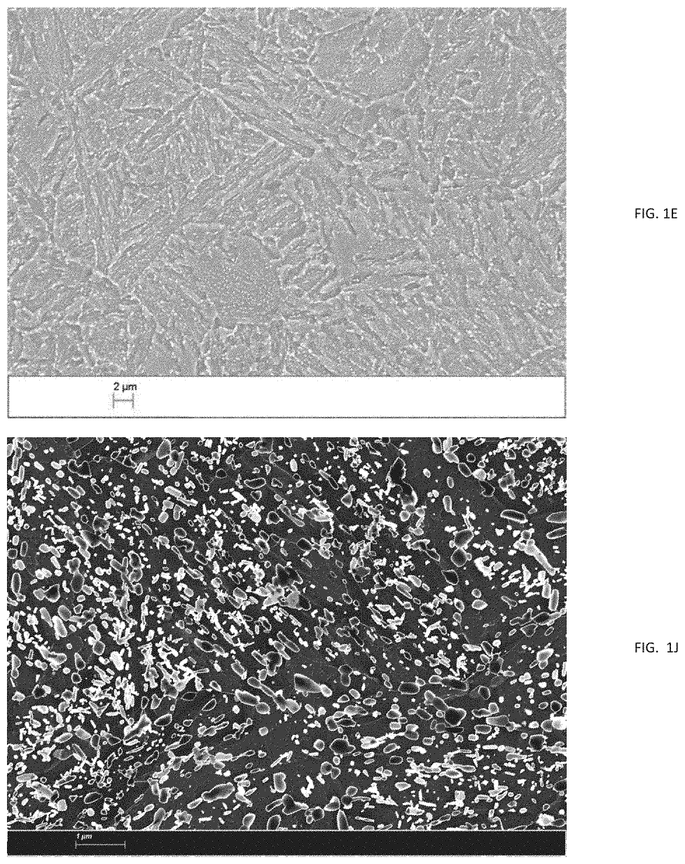

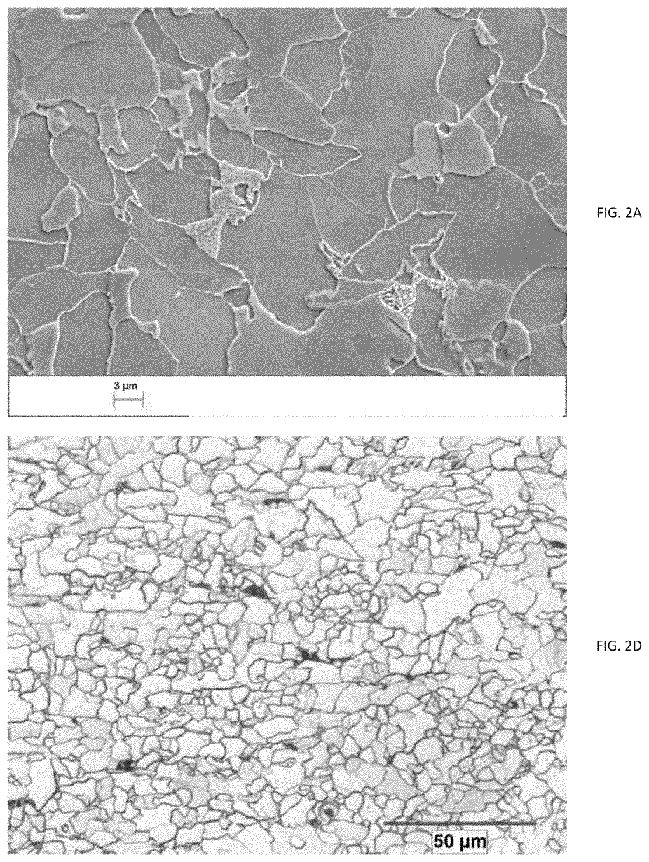

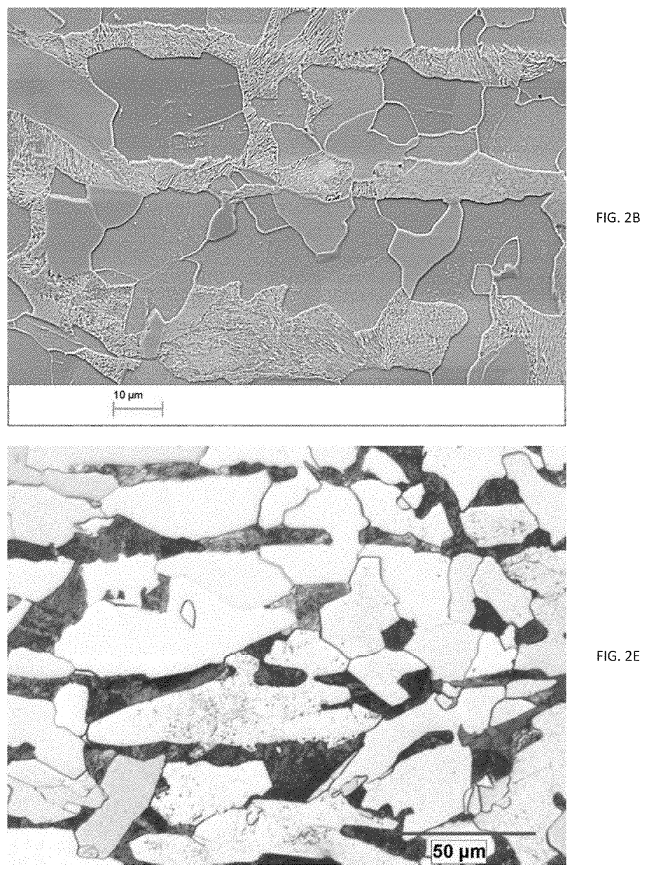

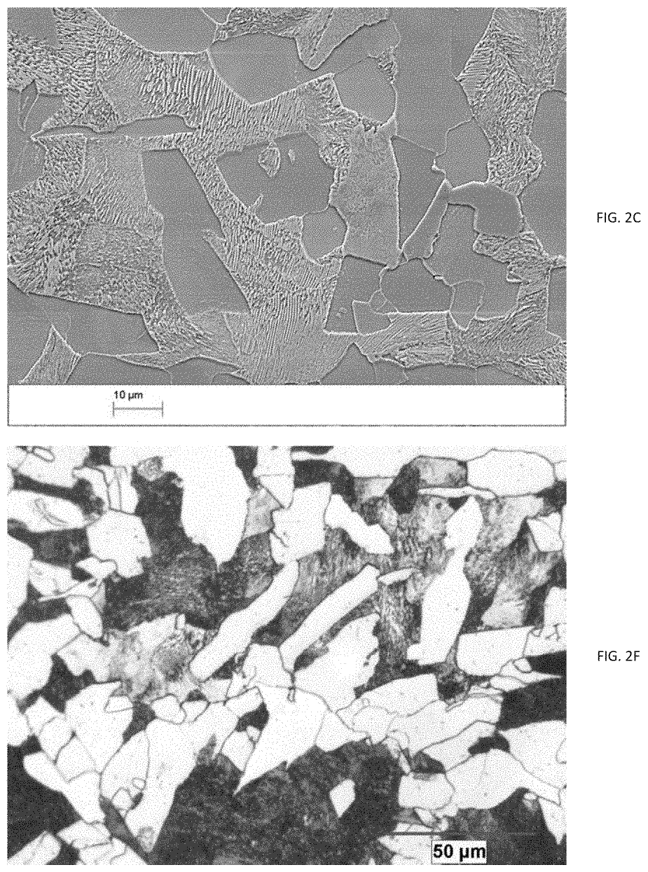

The L80 and L801Cr carbon steels (Entries 4 and 5) have a tempered martensite microstructure. The “X” grade carbon steels (Entries 1-3 and 6-8) have a ferrite and pearlite microstructure. Details of the microstructures may be seen in FIGS. 1A-1I and 2A-2F. FIGS. 1A-1I show illustrative SEM or STEM images of the carbon steels from Entries 1-5, respectively. FIGS. 2A-2F show illustrative SEM and optical microscopy images of the various X52 carbon steels from Entries 6-8, to respectively. Carbides are shown in white contrast in the SEM images. Corresponding optical microscopy (OM) images show the carbides in black contrast. As can be seen in the sets of SEM images, a considerable degree of microstructural variation exists between the carbon steel samples. The martensitic microstructures of the L80 and L801Cr (Entries 4 and 5) carbon steels are particularly distinguished from the microstructures of the remaining carbon steel samples.

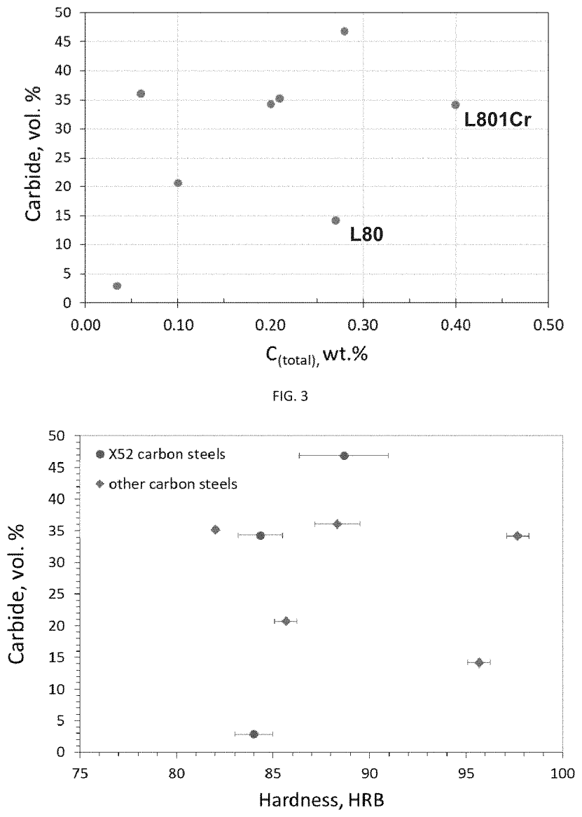

Since there are different types of microstructures within the group of carbon steel samples, no direct correlation exists between the total carbon content (Cₜₒₜₐₗ) and the volume percentage of carbides within the carbon steel samples. That is, a high total carbon content does not necessarily lead to a high volume percentage of carbides, nor does a low total carbon content necessarily lead to a low volume percentage of carbides. FIG. 3 shows a plot of total carbon content versus volume percentage of carbides for the carbon steel samples tested herein. As shown, there is no direct correlation between the total carbon content and the volume percentage of carbides in the carbon steel samples. In particular, the L80 (Entry 4) and L801Cr (Entry 5) carbon steel samples, which have a tempered martensite microstructure, are considerable outliers from the correlation trend of the “X” carbon steels (also see FIG. 8 below).

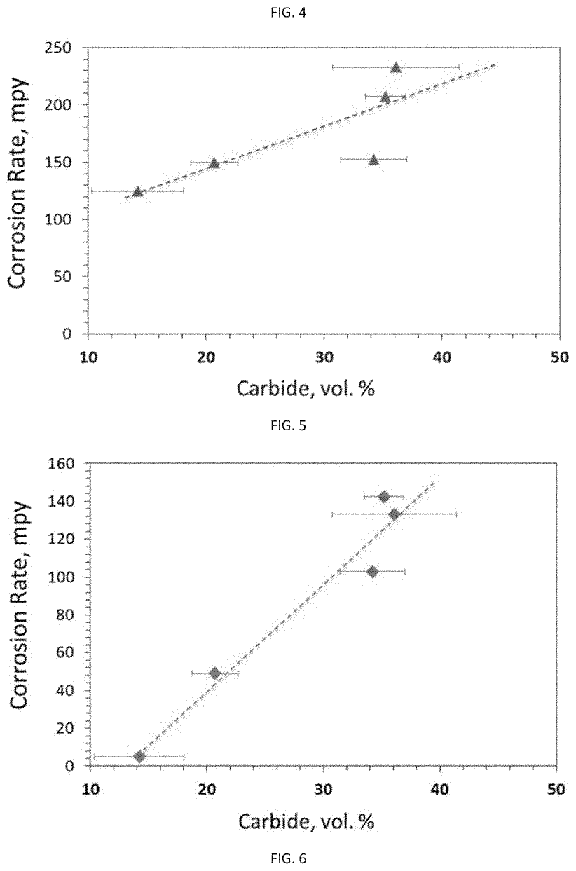

Similarly, there was no observable correlation between Rockwell Hardness of the carbon steel samples and the measured volume percentage of carbides. FIG. 4 shows a plot of B scale Rockwell Hardness versus the measured volume percentage of carbides for the carbon steel samples tested herein. Rockwell Hardness values were measured using ASTM E18-18 entitled “Standard Test Methods for Rockwell Hardness of Metallic Materials.”

Example 1: Corrosion Testing of Entries 1-5. The carbon steel samples from Entries 1-5 were tested in the same corrosion kettle for 7 days in synthetic brine at a pH of 5.1 and a temperature of 32° C. The synthetic brine had the composition listed in Table 2 below. The pH was held steady using a pH titrator.

TABLE 2

| | Concentration |
|---|---|
| Component | (mg/L) |
| NaCl | 16503 |
| Na₂SO₄ | 17 |
| NaBr | 71 |
| NaHCO₃ | 721 |
| KCl | 167 |
| CaCl₂•2H₂O | 638 |
| MgCl₂•6H₂O | 1077 |
| Acetic Acid | 596 |

The corrosion kettle was continuously charged by bubbling a CO₂/N₂ mixture having a 3 psia CO₂ partial pressure into the corrosion kettle. The shear stress at the steel sample surface was determined to be about 7-10 Pa based on multi-phase flow calculations. The weight loss from corrosion for each carbon steel sample was measured at the end of the 7-day test, and the weight loss corrosion rate (in mpy) was plotted against the volume percentage of carbides for each carbon steel sample. FIG. 5 shows a plot of volume percentage of carbides versus the weight loss corrosion rate for the carbon steel samples of Entries 1-5. As shown, the carbon steel samples with lower volume percentages of carbides exhibited the lowest corrosion rates, as measured by the weight loss corrosion rate. L80 carbon steel (Entry 4) exhibited the lowest corrosion rate, which is consistent with this carbon steel sample having the lowest volume percentage of carbides.

Moreover, there was a fairly linear correlation between the carbide volume percentages and the corrosion rates of the carbon steel samples. The one data point displaced somewhat from an approximately linear fit is that of the L801Cr sample (Entry 5). Chromium is an alloying element that is known to provide corrosion inhibiting effects. The deviation of the L801Cr data point from linearity may be due to the corrosion inhibiting effects of chromium.

The effect of the commercial corrosion inhibitor package EC1509A (Nalco) on the corrosion rates was also studied. A 3-day initial treatment of the carbon steel samples with the synthetic brine was conducted without the corrosion inhibitor being present to promote exposure of the carbides (pre-corrosion period). After the pre-corrosion period, 15 ppm of the corrosion inhibitor was added to the corrosion kettle and testing was continued for a further 7 days (10 days total). FIG. 6 shows a plot of volume percentage of carbides versus the weight loss corrosion rate for the carbon steel samples of Entries 1-5 in the presence of a corrosion inhibitor. The weight loss during the initial 3-day corrosion period was excluded from the calculations. As shown, the carbon steel samples with lower volume percentages of carbides again exhibited lower corrosion rates, and there was a reasonably linear correlation of the carbide volume percentages with the observed corrosion rates.

Therefore, among the group of tested carbon steel samples, which includes carbon steels from different performance categories and with different microstructures, L80 carbon steel (Entry 4) would be selected for its superior corrosion resistance, as determined based upon this sample having the lowest measured volume percentage of carbides.

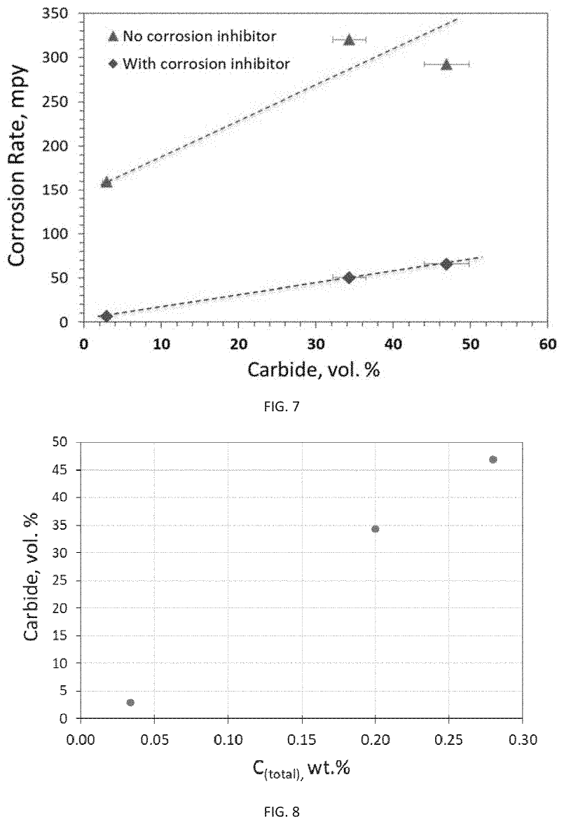

Example 2: Corrosion Testing of Entries 6-8. The carbon steel samples tested in this example were all of the X52 class, each having a different volume percentage of carbides and containing a ferrite and pearlite microstructure. Corrosion testing of the carbon steel samples from Entries 6-8 was performed under the same conditions specified in Example 1. Corrosion rates, as measured by the weight loss corrosion rate, were measured both in the presence of and in the absence of an external corrosion inhibitor. FIG. 7 shows a plot of volume percentage of carbides versus the weight loss corrosion rate for the carbon steels of Entries 6-8, both in the presence of and in the absence of a corrosion inhibitor. As shown, there was again a reasonably good correlation between the observed corrosion rate and the measured volume percentage of carbides, both in the presence of and in the absence of a corrosion inhibitor. That is, carbon steel samples having a lower volume percentage of carbides tended to have lower corrosion rates.

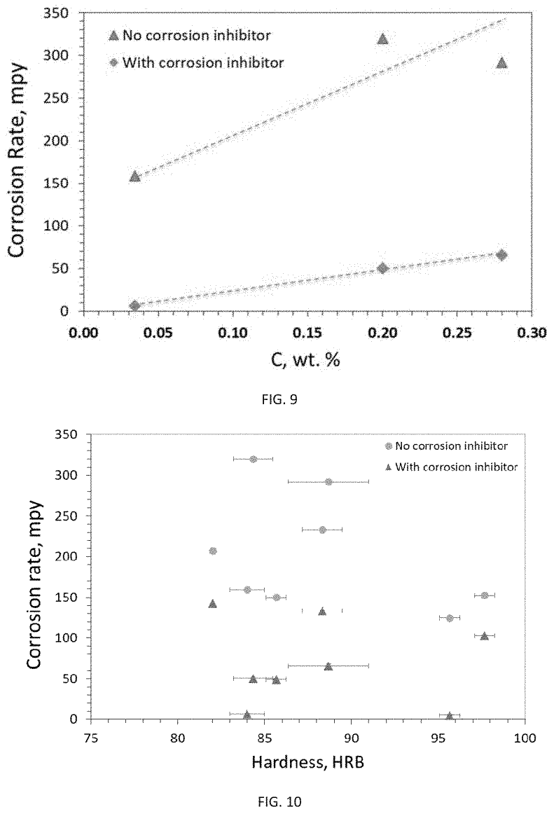

The ferrite and pearlite microstructure of the X52 carbon steel samples is thermodynamically stable. Since all of the carbon steel samples in this group have the same type of thermodynamically stable microstructure, this means that the total carbon content (Cₜₒₜₐₗ) and the volume percentage of carbides may be correlated to one another in this instance, since there is no carbide solubilization in an austenite phase. FIG. 8 shows a plot of total carbon content versus volume percentage of carbides for the X52 carbon steel samples tested in Example 2. As shown, there is a reasonably direct correlation between the total carbon content (Cₜₒₜₐₗ) and the volume percentage of carbides. FIG. 9 shows the weight loss corrosion rate data of FIG. 7 replotted against the total carbon content (Cₜₒₜₐₗ) of the X52 carbon steel samples, which demonstrate the correlation between the weight loss corrosion rate and total carbon content (Cₜₒₜₐₗ). Accordingly, when all of the carbon steel samples in a group have a thermodynamically stable microstructure, which may or may not be the same type of thermodynamically stable microstructure, the corrosion rate may be alternately correlated to the total carbon content (Cₜₒₜₐₗ) instead of the volume percentage of carbides.

Example 3: No Correlation of Hardness to Corrosion Rate. The weight loss corrosion rate data from Examples 1 and 2 was also replotted in FIG. 10 against B scale Rockwell hardness values of the carbon steel samples. As shown in FIG. 10, there was no observable correlation between the Rockwell hardness values and the weight loss corrosion rate values.

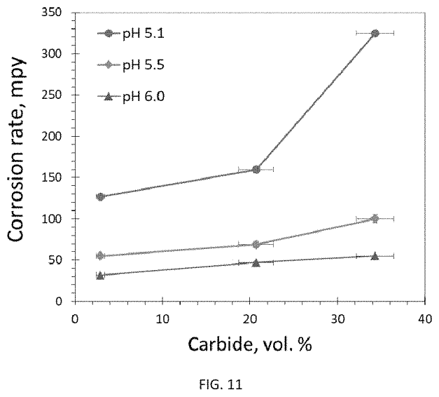

Example 4: Corrosion Rate at Different pH Values. The corrosion rate for various carbon steel samples were also measured at different pH values. FIG. 11 shows the weight loss corrosion rate data versus the volume percentage of carbides for carbon steel samples at different pH values. Corrosion testing of the samples was performed under conditions similar to those specified in Example 1, except for a different synthetic brine composition being used (Table 3) and a shorter test duration (3 days).

TABLE 3

| | Concentration | |
|---|---|---|
| Component | (g/L) | |
| NaCl | 17.15 | |
| Na₂SO₄ | 0.08 | |
| NaBr | 0.09 | |
| NaHCO₃ | 1.4 | |
| KCl | 0.11 | |
| CaCl₂•2H₂O | 0.47 | |
| MgCl₂•6H₂O | 0.6 | |
| Acetic Acid | 0.72 | (pH 6) |
| | 0.93 | (pH 5.5) |
| | 0.98 | (pH 5.1) |

As shown, there was a good correlation at pH values of 5.5 and 6.0, but at a pH value of 5.1 there was no longer a linear correlation.

All documents described herein are incorporated by reference herein for purposes of all jurisdictions where such practice is allowed, including any priority documents and/or testing procedures to the extent they are not inconsistent with this text. As is apparent from the foregoing general description and the specific embodiments, while forms of the disclosure have been illustrated and described, various modifications can be made without departing from the spirit and scope of the disclosure. Accordingly, it is not intended that the disclosure be limited thereby. For example, the compositions described herein may be free of any component, or composition not expressly recited or disclosed herein. Any method may lack any step not recited or disclosed herein. Likewise, the term “comprising” is considered synonymous with the term “including.” Whenever a method, composition, element or group of elements is preceded with the transitional phrase “comprising,” it is understood that we also contemplate the same composition or group of elements with transitional phrases “consisting essentially of,” “consisting of,” “selected from the group of consisting of,” or “is” preceding the recitation of the composition, element, or elements and vice versa.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces.

One or more illustrative embodiments are presented herein. Not all features of a physical implementation are described or shown in this application for the sake of clarity. It is understood that in the development of a physical embodiment of the present disclosure, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for one of ordinary skill in the art and having benefit of this disclosure.

Therefore, the present disclosure is well adapted to attain the ends and advantages as well as those that are inherent therein. The particular embodiments disclosed above are illustrative only, as the present disclosure may be modified and practiced in different but equivalent manners apparent to one having ordinary skill in the art and having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present disclosure. The embodiments illustratively disclosed herein suitably may be practiced in the absence of any element that is not specifically disclosed herein and/or any optional element disclosed herein.

1. A method comprising:
obtaining a group of carbon steel samples, each carbon steel sample having an unknown rate of corrosion and a microstructure;
measuring a quantity of carbides within the microstructure of each carbon steel sample; and
determining a relative order of susceptibility toward corrosion within the group of carbon steel samples based upon the quantity of carbides within the microstructure of each carbon steel sample.

2. The method of claim 1, wherein the relative order of susceptibility toward corrosion is determined with respect to an environment where there is no protective corrosion scale formed upon a surface of the carbon steel samples.

3. The method of claim 1, further comprising:
identifying or providing a set of corrosive conditions;
measuring a rate of corrosion under the set of corrosive conditions for one or more standard carbon steel samples, each standard carbon steel sample having a known or measured quantity of carbides; and
determining a rate of corrosion under the set of corrosive conditions for one or more of the carbon steel samples having the unknown rate of corrosion by comparing the quantity of carbides therein to the quantity of carbides in the one or more standard carbon steel samples and the rates of corrosion under the set of corrosive conditions for the one or more standard carbon steel samples.

4. The method of claim 1, further comprising:
identifying or providing a set of corrosive conditions;
providing one or more standard carbon steel samples, each standard carbon steel sample having a known or measured rate of corrosion under the set of corrosive conditions and a known or measured quantity of carbides; and
determining a rate of corrosion under the set of corrosive conditions for one or more of the carbon steel samples having the unknown rate of corrosion by comparing the quantity of carbides therein to the quantity of carbides in the one or more standard carbon steel samples and the rates of corrosion under the set of corrosive conditions for the one or more standard carbon steel samples.

5. The method of claim 4, wherein the corrosive conditions are sweet corrosive conditions.

6. The method of claim 4, wherein the known or measured rate of corrosion and the known or measured quantity of carbides for each of the one or more standard carbon steel samples are populated in a lookup table, fit to a calibration function, plotted in a calibration curve, or any combination thereof, and the rate of corrosion under the set of corrosive conditions for one or more of the carbon steel samples having the unknown rate of corrosion is determined based upon the quantity of carbides within the microstructure and consulting the lookup table, the calibration function, the calibration curve, or any combination thereof

7. The method of claim 1, further comprising:
identifying or providing a corrosive work environment; and
selecting a carbon steel from among the group of carbon steel samples to comprise a workpiece for deployment in the corrosive work environment based upon an anticipated deployment time of the workpiece in the corrosive work environment, and a predicted susceptibility toward corrosion or a predicted rate of corrosion of the carbon steel in the corrosive work environment, the predicted susceptibility toward corrosion or the predicted rate of corrosion being determined based upon the quantity of carbides within the microstructure of the carbon steel.

8. The method of claim 1, wherein the carbides are one or more carbides located at grain boundaries or within grains within the microstructure of the carbon steel samples.

9. The method of claim 1, wherein the quantity of carbides is measured through analyzing an image obtained by optical microscopy, scanning electron microscopy or scanning transmission electron microscopy.

10. The method of claim 1, wherein the group of carbon steel samples comprises two or more carbon steels having two or more different types of microstructures.

11. A method comprising:
obtaining a group of carbon steel samples, each carbon steel sample having an unknown rate of corrosion and a thermodynamically stable microstructure; and
determining a relative order of susceptibility toward corrosion within the group of carbon steel samples based upon a total carbon content of each carbon steel sample.

12. The method of claim 11, wherein the relative order of susceptibility toward corrosion is determined with respect to an environment where there is no protective corrosion scale formed upon a surface of the carbon steel samples.

13. The method of claim 11, further comprising:
identifying or providing a set of corrosive conditions;
measuring a rate of corrosion under the set of corrosive conditions for one or more standard carbon steel samples, each standard carbon steel sample having a known or measured total carbon content and a thermodynamically stable microstructure; and
determining a rate of corrosion under the set of corrosive conditions for one or more of the carbon steel samples having the unknown rate of corrosion by comparing the total carbon content within each of the carbon steel samples to the total carbon content in the one or more standard carbon steel samples and the rates of corrosion under the set of corrosive conditions for the one or more standard carbon steel samples.

14. The method of claim 11, further comprising:
identifying or providing a set of corrosive conditions;
providing one or more standard carbon steel samples, each standard carbon steel sample having a known or measured total carbon content and a known or measured rate of corrosion under the set of corrosive conditions, and having a thermodynamically stable microstructure; and
determining a rate of corrosion under the set of corrosive conditions for one or more of the carbon steel samples having the unknown rate of corrosion by comparing the total carbon content within each of the carbon steel samples to the total carbon content in the one or more standard carbon steel samples and the rates of corrosion under the set of corrosive conditions for the one or more standard carbon steel samples.

15. The method of claim 14, wherein the known or measured rate of corrosion and the total carbon content for each of the one or more standard carbon steel samples are populated in a lookup table, fit to a calibration function, plotted in a calibration curve, or any combination thereof, and the rate of corrosion under the set of corrosive conditions for one or more of the carbon steel samples having the unknown rate of corrosion is determined based upon the total carbon content and consulting the lookup table, the calibration function, the calibration curve, or any combination thereof

16. The method of claim 14, wherein the standard carbon steel samples all have the same type of thermodynamically stable microstructure and the thermodynamically stable microstructure of the standard carbon steel samples is the same as that within the carbon steel samples having the unknown rate of corrosion.

17. The method of claim 11, further comprising:
identifying or providing a corrosive work environment; and
selecting a carbon steel from among the group of carbon steel samples to comprise a workpiece for deployment in the corrosive work environment based upon an anticipated deployment time of the workpiece in the corrosive work environment, and a predicted susceptibility toward corrosion or a predicted rate of corrosion of the carbon steel in the corrosive work environment, the predicted susceptibility toward corrosion or the predicted rate of corrosion being determined based upon the total carbon content of the carbon steel.

18. The method of claim 11, wherein the total carbon content correlates directly with a quantity of carbides within the thermodynamically stable microstructure.

19. The method of claim 18, wherein the carbides are one or more carbides located at grain boundaries or within grains within the thermodynamically stable microstructure.

20. The method of claim 11, wherein the carbon steel samples having the unknown rate of corrosion all have the same type of thermodynamically stable microstructure.

21. A method comprising:
identifying or providing a set of corrosive conditions;
obtaining or measuring a rate of corrosion under the set of corrosive conditions for one or more standard carbon steel samples, each standard carbon steel sample having a known quantity of carbides;
obtaining or preparing a carbon steel having an unknown rate of corrosion under the set of corrosive conditions; and
determining a relative susceptibility toward corrosion or a rate of corrosion for the carbon steel under the set of corrosive conditions by comparing a quantity of carbides therein to the known quantity of carbides in the one or more standard carbon steel samples and the rates of corrosion for the one or more standard carbon steel samples.

22. The method of claim 21, wherein the relative susceptibility toward corrosion is determined with respect to an environment where there is no protective corrosion scale formed upon a surface of the carbon steel.

23. The method of claim 21, further comprising:
measuring the quantity of carbides in the carbon steel.

24. The method of claim 23, wherein the quantity of carbides in the carbon steel is measured through analyzing an image obtained by optical microscopy, scanning electron microscopy, or scanning transmission electron microscopy.

25. The method of claim 21, wherein the carbides are carbides located at grain boundaries or within grains within a microstructure of the carbon steel.
